(19) United States
(12) Patent Application Publication
(10) Pub. No.: US 2022/0062500 A1
(43) Pub. Date: Mar. 3, 2022

(54) LIGAMENT-DERIVED GRAFT AND METHODS FOR MAKING AND USING SAME IN JOINT REPAIR

(71) Applicant: Musculoskeletal Transplant Foundation, Edison, NJ (US)

(72) Inventors: David Kulber, Los Angeles, CA (US); Marc Long, Monmouth Junction, NJ (US); Matt Citro, Matawan, NJ (US)

(21) Appl. No.: 17/412,744

(22) Filed: Aug. 26, 2021

Related U.S. Application Data
(60) Provisional application No. 63/071,605, filed on Aug. 28, 2020.

(51) Int. Cl. A61L 27/36

(52) U.S. Cl. CPC A61L 27/3604; A61L 2430/24; A61L 27/3662

(57) ABSTRACT

A ligament-derived graft is provided which comprises a portion of a ligament sample recovered from a mammalian donor and which has been cut approximately in half lengthwise, where the ligament derived graft has dimensions suitable for use in a surgical procedure to repair small to mid-sized, or less load bearing joints. Small joints for which the ligament-derived grafts may be used to repair, include, interphalangeal, metacarpophalangeal joint, scapholunate joint, and other joints. In some embodiments, the dimensions of the ligament-derived graft include a length of from about 50 mm to about 150 mm, and a width of from about 3 mm to about 20 mm. In some embodiments, ligament samples used to make the ligament-derived grafts are lateral or medial collateral ligaments. Methods of using such ligament-derived grafts include attaching one or more ligaments, at their ends, to bones on opposite sides of the joint being repaired, so that the ligament(s) extend across the joint. When two grafts are used, they may extend across the joint, either on the same or opposite lateral sides of the joint.

### CROSS-REFERENCE TO RELATED APPLICATIONS

The present application claims the benefit of U.S. Provisional Application No. 63/071,605, filed Aug. 28, 2020, the entire disclosure of which is incorporated by reference herein.

### FIELD

The present invention relates to grafts derived from ligaments, as well as methods for making and using such grafts for surgical repair and reconstruction of joints.

### BACKGROUND

Joints are the point of contact or articulation between two or more bones of a subject's skeleton and are typically more or less movable within a range of motion. In addition to the portions of each bone which are in contact or articulated with one another to form a joint, the joint typically also includes one or more additional components, such as, without limitation, tendons, ligaments, cartilage, bursa, synovium and synovial fluid. Ligaments connect bone to bone and serve to add stability to a joint. Ligaments tend to be stronger and less elastic than tendons, which connect bone to muscle and provide shock absorption.

Several surgical methods and techniques, as well as various materials and devices, have been developed for repairing ligaments, as well as other aspects or components of the joint with which a damaged ligament is associated. Ligament repair may be the main purpose of a surgical procedure, but may also part of a broader procedure involving repair of additional joint components such as bone and cartilage. Joints commonly injured and treated by surgical procedures include, without limitation, shoulders, elbows, ankles, hips, knees, toes, and fingers.

Surgical repair of joints, including ligaments and other components, is generally performed to relieve pain and restore function to a joint after damage by disease (e.g., arthritis, osteomyelitis, infection), injury, or some other type of trauma. The goals for any method, material or device used for joint repair include one or more of the following: position the joint components as close as possible to their natural orientation, provide support and constraint which enables the joint components to remain in their reconstructed orientation, restore and enable range of motion as close as possible to natural range of motion prior to damage, facilitate organic tissue repair and regeneration in and around the joint, particularly in a manner which facilitates, or at least does not interfere with, the foregoing goals.

One or more ligaments and other components of a joint may be damaged by disease or trauma and, therefore, surgical repair of the joint will typically involve repair, treatment, reconstruction, replacement, or some combination of these, for all or a portion of one or more of the damaged ligaments, tendons, and/or other joint components. For example, in some cases, the surgical repair procedure may involve repair of a torn ligament or tendon, or even reattachment of a detached ligament or tendon. Sometimes, the damage to a tendon or ligament is so extensive that it cannot be repaired or reattached, and removal and replacement is necessary. Additionally, in some cases, the surgical procedure may involve replacement, remodeling, or realignment of the articular surface(s) of a joint, such as by osteotomy, resection, implantation of grafts, other materials, or implants, or some other procedure, or combination of procedures.

Ligament injuries in the hand and wrist are common reasons for pain, instability, and early degenerative joint changes. For instance, the ulnar collateral ligament (UCL) of the thumb may be ruptured following forceful abduction at the thumb metacarpophalangeal joint, leading to the nickname, skier's thumb. Additionally, injuries of the scapholunate ligament (SL) of the wrist are often ignored or missed following a fall on an outstretched hand. The proximal interphalangeal (PIP) joints of the digits can also become unstable after acute injury or chronic inflammatory arthritis. Surgical repair of any of the foregoing ligaments is often recommended to restore function and prevent altered forces across the joint that result in accelerated arthritis. In some instances, such as chronic injuries, repair may not possible due to degeneration of the tissue, and reconstruction must be performed.

Materials derived from tissues recovered from one or more donors, as well as devices including such materials, have been developed and used as grafts and implants useful for joint repair procedures. For example, it has long been known to use ligament-derived allografts (i.e., grafts made from or including ligaments, or portions thereof, which have been recovered from one or more human donors) to treat and repair damaged ligaments of the knee joint, including the anterior cruciate ligament (ACL), posterior cruciate ligament (PCL), the medial collateral ligament (MCL), and the lateral collateral ligament (LCL).

The use of tendon-derived allografts, are also known to be useful to treat and repair damaged tendons and ligaments of the knee joint, including the quadriceps tendon, as well as several of the ligament components, including the ACL, PCL, MCL and LCL. Such tendon-derived grafts are also known for use in the surgical treatment and repair of tendon of the interphalangeal and metacarpophalangeal joints of the hands and feet, elbow ligaments, for ankle stabilization, and chronic muscle tendon ruptures (e.g., of the pectoralis major muscle and the biceps muscle at the elbow). Known suitable sources for such tendon-derived grafts include, without limitation, patellar tendon, hamstring tendon, anterior and posterior tibialis tendons, peroneus longus tendon, semitendinosus tendon and gracilis tendon.

It is generally accepted that using the same type of tissue graft to repair or replace the same type of tissue as that which has been damaged (i.e., treatment of “like with like” tissue) is preferred. However, sometimes this is not possible or is impractical. For example, in some cases tendons recovered from donors do not have sufficient length or the necessary properties (e.g., tensile strength and elasticity) to effectively replace damaged tendons in a subject. Reconstruction of collateral ligaments in small joints has typically involved using other tissue such as tendons or fascia, as there is no available donor site to harvest autologous collateral ligaments from human patients because all collateral ligaments are needed and harvesting them would result in significant morbidity. Recently, synthetic materials have been employed to support collateral ligament repair (e.g., LABRALTAPE™ as previously mentioned).

In view of the ongoing need for biocompatible non-synthetic materials having characteristics specifically useful for joint repair and reconstruction, especially joints such as those in the hand, wrist, foot, ankles, elbows, and shoulders the invention described and contemplated herein provides ligament-derived grafts for use in joint repair procedures involving such joints, as well as methods for making and using such ligament-derived grafts.

### SUMMARY OF THE INVENTION

The invention described and contemplated herein relates to a ligament-derived graft comprising a portion of a ligament sample recovered from a mammalian donor and having dimensions suitable for use in a surgical procedure to repair a joint of a subject. For example, without limitation, the ligament sample may comprise a lateral collateral ligament or a medial collateral ligament. The mammalian donor of the ligament sample may, for example without limitation, be a human and, therefore, the graft would be a ligament-derived allograft.

In some embodiments, the ligament-derived graft comprises approximately 45% to 55% of the ligament sample, which was cut lengthwise, approximately in half, to produce two hemi-collateral ligaments, each of which is useful as a ligament-derived graft. In some embodiments, the ligament-derived graft of claim 1, wherein the ligament-derived graft has a length of from about 50 millimeters (mm) to about 150 mm, and a width of from about 3 mm to about 20 mm.

The joint undergoing repair may be a small joint, such as an interphalangeal joint, a metacarpophalangeal joint, a radiocarpal joint, an intercarpal joint, a metatarsophalangeal joint, an intermetatarsal joint, a tarsometatarsal joint, a talocrural joint, a subtalar joint, an inferior tibiofibular joint, and the like, or a mid-sized joint or less load bearing joint, such as a shoulder, an elbow, and the like.

A method for repairing a joint of a subject using the aforesaid ligament-derived graft is also provided and comprises attaching at least one such ligament-derived graft, at its ends, to at least two bones of the joint being repaired. More particularly, in some embodiments, the step of attaching may comprise attaching a first end of the ligament-derived graft to a first attachment point of a first bone of the joint, and attaching a second end of the ligament-derived graft to a second attachment point of a second bone of the joint. The first and second attachment points may be located on opposite sides of the joint and, after the attaching steps are performed, the ligament-derived graft extends across the joint.

In some embodiments, the method for repairing a joint of a subject includes using at least two ligament-derived grafts and the step of attaching comprises: attaching a first end of a first ligament-derived graft to a first attachment point of a first bone of the joint; attaching a second end of the first ligament-derived graft to a second attachment point of a second bone of the joint; attaching a first end of a second ligament-derived graft to a third attachment point of a third bone of the joint, which may be the same or different from either of the first and second bones of the joint; and attaching a second end of the second ligament-derived graft to a fourth attachment point of a fourth bone of the joint, which is different from the third bone but may be the same or different from either of the first and second bones of the joint.

In some embodiments of the method for repairing a joint of a subject, the first and second attachment points are located on opposite sides of the joint and, after the attaching steps are performed, the first ligament-derived graft extends across the joint; and the third and fourth attachment points are located on opposite sides of the joint and, after the attaching steps are performed, the second ligament-derived graft extends across the joint. In some embodiments, after the attaching steps are performed, the first ligament-derived graft extends across the joint on a first lateral side of the joint, and the second ligament-derived graft also extends across the joint on the first lateral side of the joint. In some embodiments, after the attaching steps are performed, the first ligament-derived graft extends across the joint on a first lateral side of the joint, and the second ligament-derived graft extends across the joint on a second lateral side of the joint, wherein the second lateral side of the joint is opposite the first side thereof.

A method for producing a ligament-derived graft having dimensions suitable for use in a surgical procedure to repair a joint of a subject, the method comprising the steps of: obtaining a ligament sample recovered from a mammalian donor; and cutting the ligament sample lengthwise, through its thickness, to produce at least a first ligament portion and a second ligament portion, at least one of which is useful as a ligament-derived graft in a surgical procedure to repair a joint. In some embodiments, the ligament sample may comprise a lateral collateral ligament or a medial collateral ligament.

In some embodiments of the method for producing a ligament-derived graft, the first ligament portion comprises approximately 40% to 70% of the ligament sample, and the second ligament portion comprises approximately 30% to 60% of the ligament sample. In some embodiments, the step of cutting comprises cutting the ligament sample approximately in half, lengthwise and through its thickness, wherein the first ligament portion is a first hemi-ligament comprising approximately 45% to 55% of the ligament sample, and the second ligament portion is a second hemi-ligament comprising approximately 45% to 55% of the ligament sample. In some embodiments, the dimensions of the ligament-derived graft comprise a length of from about 50 mm to about 150 mm, and a width of from about 3 mm to about 20 mm.

In some embodiments, of the method for producing a ligament-derived graft, the method further comprises, before or after the step of cutting, subjecting the ligament segment, or a portion thereof, to one or more processing steps selected from: cleaning; disinfecting; sterilizing; further resizing; contacting with one or more components selected from preservation media, culture media, another biocompatible fluid, a therapeutic material, cells or cell components; and packaging for storage, shipping or both.

### BRIEF DESCRIPTION OF THE FIGURES

FIG. 1 is a drawing showing a donor knee joint and the lateral and medial collateral ligaments (LCL and MCL) which are recovered therefrom for use as ligament samples;

FIGS. 2A-2B are photographs showing a first recovered medial collateral ligament sample;

FIGS. 3A-3B are photographs showing a second recovered medial collateral ligament sample;

FIG. 4 is a photograph showing a recovered MCL and the location of a cut which forms a pair of exemplary ligament-derived grafts, (i.e., hemi-collateral ligaments);

FIG. 5 is a is a photograph showing several exemplary ligament-derived grafts (i.e., hemi-collateral ligaments) produced from recovered MCLs;

FIG. 6 is a drawing of a skeletal hand which includes an interphalangeal joint (IPJ); and

FIG. 7 is a drawing showing the enlarged IPJ of FIG. 6 and the positioning and orientation of a ligament-derived graft implanted therein.

### DETAILED DESCRIPTION

Definitions

Unless stated otherwise, or implicit from context, the following terms and phrases have include the meanings provided below. Unless explicitly stated otherwise, or apparent from context, the terms and phrases below do not exclude the meaning that the term or phrase has acquired in the art to which it pertains. Unless otherwise defined, all technical and scientific terms used herein have the same meaning as commonly understood by one of ordinary skill in the art to which this invention belongs. It should be understood that this invention is not limited to the particular methodology, protocols, and reagents, etc., described herein. The definitions and terminology used herein are provided to aid in describing particular embodiments and are not intended to limit the scope of the claimed invention.

Unless stated otherwise, the terms “a” and “an” and “the” and similar references used in the context of describing a particular embodiment of the application (especially in the context of claims) can be construed to cover both the singular and the plural.

“Beneficial results” or “desired results” may include, but are in no way limited to, lessening or alleviating the severity of the disease condition, preventing the disease condition from worsening, curing the disease condition, preventing the disease condition from developing, lowering the chances of a patient developing the disease condition, decreasing morbidity and mortality, and increasing a subject's quality of life. As non-limiting examples, “beneficial results” or “desired results” may be alleviation of one or more symptom(s), diminishment of extent of the deficit, stabilized (i.e., not worsening) state of a joint condition, delay or slowing of a joint condition, and amelioration or palliation of symptoms associated with a joint condition.

The term “cartilage,” as used herein, includes osteochondral cartilage, as well as non-articulating cartilage. As used herein, “osteochondral cartilage” means load-bearing cartilage which is present or recovered from a joint such as knees, shoulders, hips, elbows, and the like. As used herein, “non-articulating cartilage” means non-load-bearing cartilage such as meniscal tissue, larum, or that which is present or recovered from body features other than joints such as ear lobes, nose, ribs, and the like.

The terms “diseases”, “conditions,” and “disease conditions,” as used herein may include, but are in no way limited to any form of joint-related condition, disease or disorder, for example, damaged joints and arthritic joints.

The term “graft,” as used herein, means a biocompatible material used for implantation in, or other placement in or on, a subject (e.g., a patient). Additionally, the term “implant” as used herein has the same meaning as the term “graft” and these terms are used interchangeably. Some grafts are made from or otherwise include tissue matrices produced by processing tissue samples recovered from one or more donors, either live or deceased. The donor(s) and the receiving subject are both animals, but need not be the same type or species of animal. Accordingly, each of the donor(s) and the receiving subject may, independently, be any type of mammal (including humans and non-human mammals), reptile, amphibian, fish, or bird. Furthermore, “grafts” include, but are not limited to, those including material derived from tissue transferred from one body site to another in the same individual (“autograft”), material derived from tissue transferred between genetically different members of the same species (“allograft”), and material derived from tissue transferred between different species (“xenograft”).

The terms “length,” “width,” and “thickness” are used herein to describe the dimensions of ligaments samples recovered from donors, as well as the dimensions of the ligament-derived grafts produced from those samples, wherein “length” is the greatest dimension, “width” is the next greatest dimension, and “thickness” is the least or smallest dimension of the ligament sample, as well as of the graft produced therefrom.

As used herein, the term “joint” is used to mean a joint in any of the hand, wrist, foot, ankle, shoulder, and elbow of a subject that are generally small to mid-sized or less load bearing joints (e.g., as compared to knees and hips). In addition to the portions of the bones which form a joint, a joint typically also includes one or more additional components, such as, without limitation, ligaments, tendons, cartilage, bursa, synovium and synovial fluid. Without limitation, representative examples of particularly small joints include: interphalangeal, metacarpophalangeal, radiocarpal, intercarpal, metatarsophalangeal, intermetatarsal, tarsometatarsal, talocrural, subtalar, and inferior tibiofibular joints. However, as noted above, the presently described and contemplated ligament-derived grafts and methods for their use are not limited to repair and reconstruction of the smallest joints.

As used herein, the term “repair” means surgical treatment of a damaged, atrophied, weak, worn, or diseased joint, including any one or more components thereof, to restore, in so far as possible, the health, structure, function, and range of motion of the joint, by (without limitation) repositioning, reconstructing, replacing (in whole or in part) the joint or its components, and any combination thereof. For example, without limitation, a procedure to repair a joint may involve reconstructing, reinforcing or replacing of one or more ligaments or tendons of the joint.

As used herein, a “subject” means a human or non-human animal. Usually the animal is a mammalian vertebrate such as, without limitation, primate, rodent, porcine, bovine, canine, feline, equine, etc., but may also be a reptile a reptile, an amphibian, a fish, and/or a bird. The nature of the animal is not limited and, therefore, the animal may be domestic, companion, wild, feral, etc. The terms, “patient”, “individual” and “subject” are used interchangeably herein. While human, non-human primate, dog, cat, pig, cow, horse, mammal species are preferred subjects, they are not limited to these examples.

A subject can be one who has been previously diagnosed with or identified as suffering from or having a condition in need of treatment (e.g., a damaged ligament, arthritic joint, etc.) or one or more complications related to the condition, and optionally, have already undergone treatment for the condition or its complications. Alternatively, a subject can also be one who has not been previously diagnosed as having a condition or one or more complications related to the condition. For example, a subject can be one who exhibits one or more risk factors for a condition or one or more complications related to the condition or a subject who does not exhibit risk factors. A “subject in need” of treatment for a particular condition can be a subject suspected of having that condition, diagnosed as having that condition, already treated or being treated for that condition, not treated for that condition, or at risk of developing that condition.

As used herein, the terms “treat,” “treatment,” “treating,” or “amelioration” when used in reference to a disease, disorder or medical condition, refer to both therapeutic treatment and prophylactic or preventative measures, wherein the object is to prevent, reverse, alleviate, ameliorate, inhibit, lessen, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a disease, disorder or medical condition is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results, or lower the chances of the individual developing the condition even if the treatment is ultimately unsuccessful. Those in need of treatment include those already with the condition as well as those prone to have the condition or those in whom the condition is to be prevented.

Rationale/Need for Invention

Currently, the majority of ligamentous reconstructions in the hand, when involving graft material, use autograft tendon either by way of free tendon transfer or local split tendon grafting. Use of tendon to reconstruct ligament structures is based on necessity and historical precedent. Tendons provide the length required to recreate a ligament, with enough excess to thread through bone tunnels and anchor to achieve reliable fixation. Due to redundancy within muscular compartments, tendons can also be harvested and repurposed without significant loss of function at their original location.

Nonetheless, use of tendon autograft has some inherent disadvantages, including donor site morbidity and altered material properties compared to the ligaments they are intended to reconstruct. Tendons contain more collagen, less elastin, and are less viscoelastic than ligaments, which may lead to the attenuation and failure of some reconstructions over time. In other words, for example, when used for ligamentous repair, tendon-derived grafts tend to stretch over time and, thereby, fail to provide the structure and dimensions required to effectively function in place of damaged, atrophied, weak, worn, or diseased collateral tendons. Given these shortcomings, new techniques to replicate biomechanics and native ligamentous anatomy, coupled with investigation of alternative materials, are currently under way.

The use of allograft ligaments for ligamentous reconstruction in the hand and wrist has not yet been well studied or developed. Although use of PIP collateral ligaments and SL ligaments for SL repairs with bone-PIP collateral ligament-bone composite collateral ligament grafts has been studied, PIP collateral ligaments are small, and thus may not be suited to reconstruction of larger ligaments. Due to the ongoing need to repair and reconstruct joints and ligaments of a wide range of sizes, there remains a need for adequately sized ligament material which would for sufficient resizing, as well as methods for their preparation and use in such procedures.

Fresh frozen ligament allograft prepared from ethically recovered ligaments of deceased donors for example, would provide an alternative option for upper extremity reconstruction. By using grafts made from allograft ligament instead of autograft tendon, donor site morbidity will be eliminated, surgical time required to otherwise harvest tendon autograft during the repair/reconstruction procedure will be eliminated, and the graft may confer mechanical advantages owing to its material similarities to the native ligament being reconstructed (i.e. a ligament graft for a ligament reconstruction). Particularly for reconstruction of PIP collateral ligaments, thumb UCL, and SL ligaments, a number of techniques and grafts, generally tendon autografts, are used. Using grafts prepared from recovered collateral knee ligaments, represents a novel technique and a treatment alternative to previously described surgical reconstructions.

There exist a myriad of reconstruction techniques and—when grafts are utilized—various graft sources for reconstruction of injured SL ligaments, PIP collateral ligaments, thumb UCLs and elbow UCLs (i.e., Tommy John Surgery). Studies have shown varying efficacies in outcome for many of these techniques. For example, reconstruction techniques have been described for SL ligaments and include capsulodesis and various tenodesis techniques using flexor carpi radialis (FCR) or extensor carpi radialis brevis (ECRB) tendon graft, bone-extensor retinaculum-bone grafts from the distal radius, among others. See, Crawford K, et al., Ligament Reconstruction: A Critical Analysis Review, JBJS Rev. 2016; 4(4):e41-8. In PIP joint collateral ligament reconstruction, techniques have been described using palmaris longus (PL) and flexor digitorum superficialis (FDS) tendon autografts. See, Carlo J, et al., Collateral Ligament Reconstruction of the Proximal Interphalangeal Joint, J Hand Surg Am. 2016; 41(1):129-32. For thumb UCL reconstruction, techniques include capsulorrhaphy, adductor aponeurosis advancement, transfer of extensor indicus proprius (EIP), extensor policis brevis (EPB), abductor policis longus (APL), or PL tendons. See, e.g., Rhee P C, et al., Management of thumb metacarpophalangeal ulnar collateral ligament injuries, J Bone Joint Surg Am. 2012; 94(21):2005-12; Samora J B, et al., Outcomes after injury to the thumb ulnar collateral ligament—a systematic review, Clin J Sport Med. 2013; 23(4):247-54; and Pulos N, et al., Treatment of Ulnar Collateral Ligament Injuries of the Thumb: A Critical Analysis Review, JBJS Rev. 2017; 5(2). Various measured outcomes have been produced with many of these techniques. Given so many different options and reported outcomes for currently available techniques and grafts for tendon repair and reconstruction, there is no universally agreed-upon gold-standard technique(s) for any of the above-discussed and similar injuries involving tendons.

The use of ligament-derived grafts, including allografts, are expected to provide several benefits and improvements over existing grafts and techniques for tendon repair and reconstruction, including, for example, without limitation, improved pain reduction and greater, joint stability, function, range of motion, and strength of repaired and reconstructed joint(s).

Ligament-Derived Graft and Method for Making

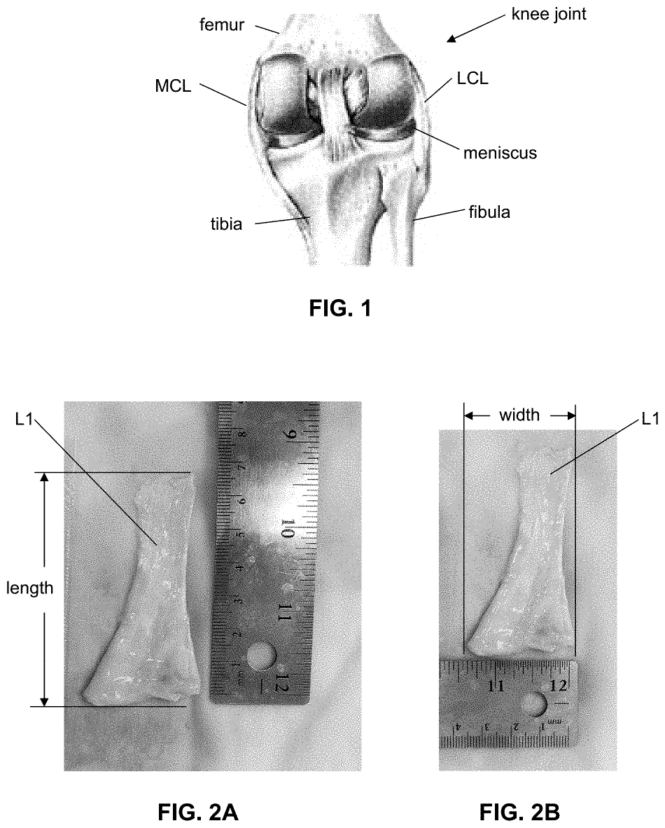

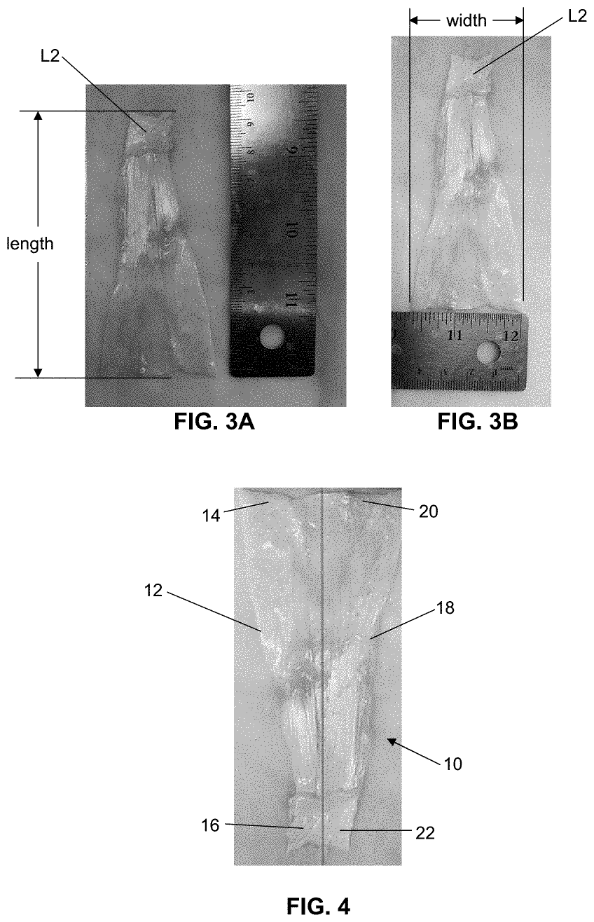

A ligament-derived graft is a graft which is produced from, or includes material produced from, a ligament sample recovered from a donor. The particular type of ligament sample is not limited, however, as will be recognized by persons of ordinary skill in the relevant art, the ligament sample should be of sufficient size and shape that it will have sufficient length and width to fit into the intended location of implantation in or proximate a joint in need of repair. For example, without limitation, where the damaged joint to be repaired is an interphalangeal or metacarpophalangeal joint, then a medial or lateral collateral ligament (LCL or MCL, see FIG. 1) would be a suitable ligament sample for producing the ligament-derived graft. More particularly, as shown in FIG. 1, the femur, tibia and fibula bones of a leg are joined together at the knee joint, which further includes a meniscus comprising cartilage, and the LCL and MCL ligaments. The lengths of LCL and MCL ligaments recovered from adult human donors typically range from about 55 to about 90 millimeters. For example, FIGS. 2A-2B and 3A-3B show two recovered MCL ligament samples L1, L2 and, as is apparent from these figures, each ligament sample has a length which is clearly its greatest dimension, as compared to its width (and thickness, not shown per se).

Collateral ligaments have unique intrinsic properties in tensile strength compared to other tissues. All joints have collateral ligaments and sometimes they rupture due to trauma or wear down and are contribute to arthritic conditions, pain and discomfort. The use of collateral ligaments recovered from deceased donors for repair of collateral ligaments in joints will replace like tissue with like tissue by restructuring damaged collateral ligaments in patients using bone tunnels, interference screws, bone anchors, etc.

As shown in FIG. 4, in accordance with the method described and contemplated herein for producing a ligament-derived grafts, after recovery from a donor, a ligament sample 10 is cut (e.g., using a device with a blade such as a scalpel, knife), lengthwise and in a substantially straight line, to produce at least two portions, i.e., at least a first portion 12 and a second portion 18. In some embodiments, the first portion 12 may comprise approximately 40% to 70% of the ligament sample, while the second portion would comprise the remaining approximately 30% to 60% of the ligament sample, respectively. Depending on the joint or joints to be repaired, each of the first and second portions 12, 18 of the ligament sample would be useful as a ligament-derived graft for repairing a joint or joints.

In some embodiments a ligament sample 10 is cut approximately in half, lengthwise in a substantially straight line to produce two halves, or “hemi” collateral ligaments (“HCLs”) 12, 18, each of which is a ligament-derived graft suitable for use in repair of a joint, such as an interphalangeal or metacarpophalangeal joint. In such circumstances, a first one of the resulting HCL ligament-derived grafts comprises approximately 45% to 55% of the ligament sample, while the second one of the HCL ligament-derived grafts comprises the remaining approximately 45% to 55%, respectively, of the ligament sample.

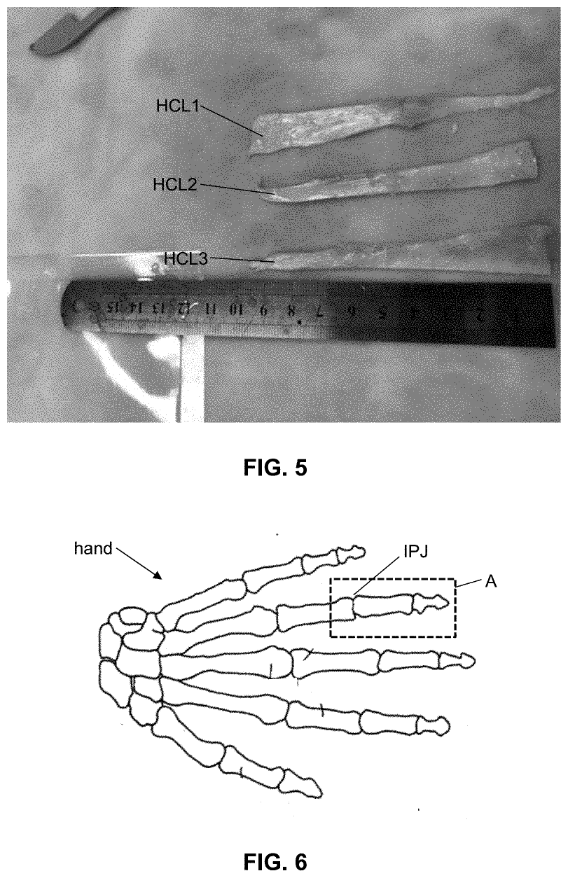

It is noted that the first ligament-derived graft 12 has first and second ends 14, 16, each of which will be secured to a respective first and second bones of a joint (i.e., so as to extend across the joint) undergoing repair, as described in more detail below. Similarly, the second ligament-derived graft 18 has first and second ends 20, 22 useful for the same purpose. FIG. 5 shows several such ligament-derived grafts HCL1, HCL2, HCL3 (i.e., hemi-collateral ligaments) in accordance with the graft and method described and contemplated herein.

In some embodiments, suitable dimensions for a ligament-derived graft (e.g., each “hemi” collateral ligament, HCL, 12, 18) which would be useful for repair of an interphalangeal or metacarpophalangeal joint are: a length of from about 40 to about 150 millimeters (mm), a width of from about 18 to about 30 mm, and a thickness of from about 1 to about 2 mm. Accordingly, the resulting ligament-derived grafts 12, 14 should have dimensions of: a length of at least about 40 mm (i.e., same as the ligament sample 10) and up to about 150 mm (15.0 cm), and a width of from about 3 to about 20 mm (i.e., about half the width of the ligament sample 10). While there is no particular limitation on the thickness of the ligament-derived graft, other than generally being less than the width, typical thicknesses are from about 1 to about 2 mm (i.e., same as the ligament sample 10).

After the ligament sample 10 is cut to produce the two “hemi” collateral ligaments (“HCLs”), one or both of the HCLs is typically subjected to one or more processing steps including, without limitation: cleaning; disinfecting; sterilizing; further resizing; contacting with one or more components such as preservation media, culture media, or another biocompatible fluid, therapeutic materials such as, without limitation, growth factors, disinfectants, hyaluronic acid or its derivatives, cells or cell components; and packaging for storage, shipping or both. In some embodiments of the method for producing a ligament-derived graft, an HCL is subjected to one or more disinfecting steps which involve soaking the HCL in one or more antibiotic-containing solutions, which may be the same or different from one another.

In some embodiments of the method for producing a ligament-derived graft, an HCL is subjected to one or more processing steps each of which is performed under aseptic conditions, whereby no terminal sterilization is necessary or performed. In some embodiments of the method for producing a ligament-derived graft, an HCL is subjected to a terminal sterilization step which is performed after all other processing steps. Sterilization may be performed by any effective technique known now or in the future to persons of ordinary skill in the relevant art including, but without limitation, exposure to gamma radiation, electron beam (e-beam), or ultraviolet radiation, contact or exposure to chemical agents (e.g., alcohol, phenol, ethylene oxide gas, acids, bases, or peroxides), heat, and the like, for sufficient duration and at effective dosages.

It is noted that, although the foregoing discussion describes recovery and use of LCL and MCL ligaments to produce the ligament-derived grafts, as already mentioned, the type of ligament recovered for use as the ligament sample is not limited and suitable ligaments are not intended to be limited by this description. It is well within the ability of persons of ordinary skill in the relevant art to select a type of ligament to be recovered and used as the ligament sample based on the particular joint intended to be treated with repair using the resulting ligament-derived graft.

The ligament-derived grafts are useful for stabilizing hand digits where deficiencies in endogenous tendons and ligaments exist. For example, without limitation, a ligament may be too weak to effectively perform its function in a joint, or a ligament may be torn or even detached. In some cases, the damage to a ligament is so extensive that it cannot be repaired or reattached, and removal and replacement is necessary. Previously employed solutions involve the use of sutures or synthetic materials (e.g., LABRALTAPE™). A biological (i.e., non-synthetic) solution, such as allograft material is preferred. Thus, the ligament-derived grafts described and contemplated herein, provides such a solution not previously available.

Surgical Repair—Method of Use

One exemplary embodiment of the method for using the ligament-derived grafts described above for repair of a joint generally comprises: (1) surgically exposing a damaged joint; (2) removing any damaged ligament, damaged tendon, or portions thereof, from the intended location for affixing the ligament-derived graft; (3) positioning a ligament-derived graft and affixing each end thereof to a respective bone of the joint (i.e., each end is affixed to a bone at an attachment point), such that the graft extends across the joint; and (4) closing the reconstructed finger joint.

The step of positioning the ligament-derived graft may include positioning and orienting the graft to extend across the joint. The techniques for affixing the ends 14, 16 of the ligament-derived graft 12 to the bones of the joint are not particularly limited and include any such techniques for securing grafts to bone known now or in the future to persons of ordinary skill. For example, without limitation, securing the ends 14, 16 of the ligament-derived graft may be accomplished using bone tunnels, interference screws, or suture anchors.

Furthermore, in some embodiments, more than one (i.e., 2, 3 or 4) ligament-derived grafts may be positioned and their ends affixed to the bones of the joint (i.e., at several respective attachment points), so that two or more ligament-derived grafts extends across the joint proximate to one another (e.g., on the same lateral side of the joint). In some embodiments, a first one or more ligament-derived grafts may be positioned, oriented and secured to the bones by their ends on one lateral side of the joint, and a second one or more ligament-derived grafts may be positioned, oriented and secured to the bones by their ends on an opposite lateral side of the joint. It is contemplated that, in some embodiments, two or more ligament-derived grafts may be combined by joining or attaching them end to end, or having overlapping portions, to provide a composite ligament-derived graft with a greater length, as may be required in some cases. In such embodiments, a composite ligament-derived graft will be placed across a joint and attached to a first bone of the joint by one end of one of the constituent ligament-derived grafts, and to a second bone of the joint by an opposite, distal end of another constituent ligament-derived grafts.

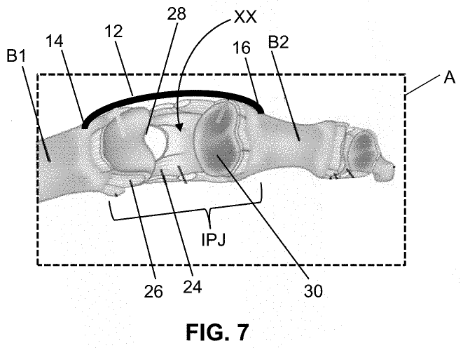

More specifically, FIG. 6 is a drawing of a skeletal hand with dotted lines to show a portion A of the hand which includes a joint, such as a proximal interphalangeal joint (proximal IPJ) in need of repair. FIG. 7 shows an enlarged view of a portion A of the hand which includes the IPJ and the location and orientation for placement of the ligament-derived graft 12 thereon. In accordance with the method for using the ligament-derived graft 12 for repair of the proximal IPJ shown in FIGS. 4 and 5, after the IPJ is exposed and any tissue or damaged tendon and ligament (such as the check-rein or collateral ligaments 24, 26, see FIG. 5) is removed, the ligament-derived graft 12 is placed and aligned across the IPJ, from a first bone B1 (such as the proximal phalanx, see FIG. 5) to a second bone B2 (such as the middle phalanx, see FIG. 5) of the IPJ. Accordingly, in this particular embodiment, the first end 14 of the ligament-derived graft 12 is secured to the proximal phalanx B1, and the second end 16 is secured to the middle phalanx B2 of the IPJ. As will be recognized by persons of ordinary skill in the relevant art, where the ligament-derived graft 12 has a length too great to fit the size of the joint to be repaired, the ligament-derived graft 12 can be further resized (e.g., cut, folded, etc.) to more efficiently fit the joint being repaired, before or after being secured by its ends 14, 16 to the first and second bones B1, B2, respectively. Other joints may also be repaired using the ligament-derived graft, such as (without limitation) the ulnar collateral ligament (UCL) of a thumb, or ligaments in any of the joints discussed and listed above.

In some embodiments, repair of a joint using a ligament-derived graft 12 may be combined with a repair procedure which involves use and implanting of a second ligament-derived graft, another type of graft or implant, or both. For example, without limitation, various types of cartilage-derived grafts have been developed and used for treatment and repair of knee joints, ankle joints, shoulder joints, and hip joints. Known cartilage-derived grafts include morselized cartilage in a paste form, cartilage-derived extracellular matrix, and reshaped pieces of cartilage. In particular, U.S. Patent Application Publication No. 2018/0140425 (hereinafter, US2018/0140425), which is hereby incorporated herein in its entirety, describes a surgical procedure using a cartilage-derived graft for treating and repairing a damaged joint in a hand or finger (e.g., radiocarpal, metacarpophalangeal, and interphalangeal joints). The procedure described in US2018/0140425 involves using a cartilage-derived graft (e.g., a meniscus-derived graft) and may be modified and improved by also using and implanting one or more ligament-derived grafts in the same joint.

In some embodiments, such a repair procedure would generally comprise the steps of: (1) opening a damaged joint (e.g., metacarpophalangeal and interphalangeal joints); (2) preparing an articular surface of the finger joint (for example, removing remaining or damaged cartilage, decorticating a first bone of the joint to expose its medullary cortex, and using reamers to create a cup and/or cup joint configurations); (3) preparing the lateral sites on the first and second bones of the joint for securing the ligament-derived graft (e.g., by removing any damaged ligament, damaged tendon, or portions thereof, from the intended location for affixing the ligament-derived graft); (4) positioning and securing one or more (e.g., 1, 2, 3, 4, 5) meniscus-derived grafts onto the prepared articular surface; (5) positioning a ligament-derived graft and affixing each end thereof to a respective bone of the joint, such that the graft extends across the joint and (6) closing the reconstructed finger joint. More than one meniscus-derived graft may be used. The meniscus-derived graft may be secured by any suitable technique known now or in the future to persons of ordinary skill including, without limitation, with sutures, anchors, adhesive, fibrin glue, screws, staples, pins, etc. Optionally, fibrin glue can be used to coat the meniscus-derived graft(s) and articular surfaces. The implanted meniscus-derived graft(s) fit neatly into the repaired joint, is revascularized, and gets incorporated into the joint as blood flows through. Range of motion is also expected to be restored.

The cartilage-derived graft described in US2018/0140425 is made of meniscus tissue which was recovered from a donor and then dimensioned (i.e., resized and shaped) to cover at least a portion of the surface of a bone in the damaged joint to be treated. One or more such cartilage-derived grafts are inserted in between the bones of the damaged joint and each is secured (e.g., with sutures, anchors, adhesive, fibrin glue, screws, staples, pins, etc.) to the articular surface of a respective one of the bones of the joint. In cases where one or more of the tendons or ligaments of the hand or finger joint are in need of repair or replacement, sometimes suture material, such as LABRALTAPE™ (commercially available from Arthrex of Naples, Fla., U.S.A.), is used to connect the bones of the joint, outside the joint, along either or both of the external lateral sides of the joint where natural tendons or ligaments would have been located to provide strength to the joint.

For example, in an exemplary embodiment, as described in US2018/0140425, a meniscus-derived graft (not shown per se) may be produced from and comprise a portion of meniscus recovered from a donor (see, e.g., FIG. 1), and a portion of meniscus is dimensioned to cover a surface of a bone (e.g., the first bone B1, the second bone B2, or both B1, B2, which are shown in FIG. 7) in the joint undergoing repair. In other embodiments, the implant may consist of or consist essentially of a portion of a meniscus, and the portion of meniscus is dimensioned to cover a surface of a bone (e.g., the first bone B1, the second bone B2, or both B1, B2, see FIG. 7) in the joint. In various embodiments, the surface is an articular surface.

For example, where the joint being repaired is a proximal IPJ, a meniscus-derived graft (not shown per se) would be inserted and positioned in the IPJ, in between the proximal phalanx B1 and middle phalanx B2, and secured to at least one of the bones B1, B2 of the IPJ (which are shown in FIG. 7). Furthermore, in such embodiments, the portion of meniscus would be dimensioned to produce a meniscus-derived graft which is sized and shaped to cover all or at least a portion of the head 28 of the proximal phalanx B1 or all or at least portion of the base 30 of the middle phalanx B2 (which are shown in FIG. 7). In some embodiments, at least two meniscus-derived grafts (not shown per se) may be used, wherein a first meniscus-derived graft is dimensioned to cover all or at least a portion of the head 28 of the proximal phalanx B1, and a second meniscus is dimensioned to cover all or at least a portion of the base 30 of the middle phalanx B2 (which are shown in FIG. 7). The step of dimensioning the portion of meniscus may, for example without limitation, involve shaping the portion of meniscus by cutting, trimming, shaving, molding, etc.

In some embodiments, the cartilage-derived graft is a meniscus-derived graft which comprises a piece of tissue harvested from a meniscus, and the piece of tissue is dimensioned to repair an osteochondral damage or defect. For example, when a portion of a subject's cartilage is removed due to the osteochondral damage or defect, the cartilage-derived graft can be used to replace portion of the removed cartilage.

In various embodiments, the portion of meniscus is shaped to be round or generally round, oval or generally oval, square or generally square, or rectangular or generally rectangular. In certain embodiments, the portion of meniscus is shaped to be flat, saucer-shaped or cup-shaped. In various embodiments, the portion of meniscus is dimensioned to be about 0.2-4.0 cm². In various embodiments, the portion of meniscus is dimensioned to be about 0.2-0.3, 0.3-0.4, 0.4-0.5, 0.5-0.6, 0.6-0.7, 0.7-0.8, 0.8-0.9, 0.9-1, 1-2, 2-3, or 3-4 cm².

In various embodiments, the portion of meniscus has a dimension (e.g., diameter, diagonal, width, or length) of about 0.2-4.0 cm. In various embodiments, the portion of meniscus has a dimension (e.g., diameter, diagonal, width, or length) of about 0.2-0.3, 0.3-0.4, 0.4-0.5, 0.5-0.6, 0.6-0.7, 0.7-0.8, 0.8-0.9, 0.9-1, 1-2, 2-3, or 3-4 cm. In some embodiments, the portion of meniscus has dimensions of about 0.5 cm×0.5 cm, 0.5 cm×1 cm, 0.5 cm×2 cm, 0.5 cm×3 cm, 0.5 cm×4 cm, 1 cm×1 cm, 1 cm×2 cm, 1 cm×3 cm, 1 cm×4 cm, 2 cm×2 cm, 2 cm×3 cm, 2 cm×4 cm, 3 cm×3 cm, 3 cm×4 cm, or 4 cm×4 cm. In various embodiments, the portion of meniscus has a thickness of about 0.1-1.0 cm. In various embodiments, the portion of meniscus has a thickness of about 0.1-0.2, 0.2-0.3, 0.3-0.4, 0.4-0.5, 0.5-0.6, 0.6-0.7, 0.7-0.8, 0.8-0.9, or 0.9-1 cm. The thickness of the portion of meniscus may be uniform or non-uniform. For example, for a saucer-shaped portion of meniscus, the center may be thicker than the edges thereof.

It is noted that the recovered meniscus sample may need to be trimmed to produce the various meniscus-derived grafts described above. In some cases, the recovered meniscus sample may be trimmed with a machine to produce perfectly round, oval, square or rectangular grafts. In other cases, however, the recovered meniscus sample may be trimmed by hand to produce grafts that are generally round, oval, square or rectangular, while the hand-cut implants could have serrated lines, sides and edges.

In some embodiments of the method, where one or more meniscus-derived grafts are used, in addition to one or more ligament-derived grafts, to perform the repair procedure, one or more meniscus-derived grafts may be secured on the surface of a first bone B1 of the joint being repaired, or on the surface of the second bone B2. With reference to FIG. 5, in some embodiments in which the joint is a proximal IPJ, in addition to securing the ends 14, 16 of a ligament-derived graft 12 to the first and second bones B1, B2, respectively, of the joint, one or more meniscus-derived grafts (not shown per se) may be secured on the surface of the head 28 of the proximal phalanx B1 of the IPJ, or on the surface of the base 30 of the middle phalanx B2. In some such embodiments, a first one or more meniscus-derived grafts (not shown per se) may be secured on the surface of the head 28 of the proximal phalanx B1 of the IPJ, and a second one or more meniscus-derived grafts (not shown per se) may be secured on the surface of the base 30 of the middle phalanx B2 (which are shown in FIG. 7).

The recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is used herein to indicate a non-limiting example and, therefore, is synonymous with the term “for example, without limitation.”

It will be understood that the embodiments of the present invention described hereinabove are merely exemplary and that a person skilled in the art may make variations and modifications without departing from the spirit and scope of the invention. All such variations and modifications are intended to be included within the scope of the present invention. Furthermore, no language in the specification should be construed as indicating any non-claimed element essential to the practice of the invention described and contemplated herein.

### EXAMPLE 1

### PIP Collateral Ligament Reconstruction

Ten patients with isolated injuries to the PIP collateral ligaments, for whom non-operative management has failed, and who have persistent instability, will undergo ligament reconstruction using allograft knee collateral ligaments (i.e., ligament-derived grafts in accordance with the invention described and contemplated herein, “HCL”). Post-operative efficacy will be determined using functional outcome scores, measured strength, range of motion, and radiographs.

The PIP collateral ligament reconstruction technique will be as follows. A longitudinal mid-axial incision is made over the ulnar/radial aspect of the PIP joint of the finger. The injured collateral ligament is debrided. Bicortical drill holes are made parallel to the joint at the base of the middle phalanx and head of the proximal phalanx. The HCL will be trimmed to the specifications of the native collateral ligament. Vicryl passing sutures on each end are threaded through the drill holes using a Hewson suture passer. An impedance screw is used in the distal bicortical drill hole (at base of middle phalanx) to fix the graft distally. The proximal portion of the graft is secured at the proximal bicortical drill hole (at the head of the proximal phalanx) with another impedance screw after tensioning the ligament in 30 degrees of flexion.

Efficacy of the foregoing procedure and the HCL (allograft knee collateral ligament) will be assessed via several modalities at the following post-operative time points: 6 weeks, 6 months, and 12 months, and will be compared to pre-operative assessments of the same modalities. DASH (Disabilities of the Arm, Shoulder & Hand) and VAS pain (Visual Analog Scale for pain) scores will be obtained with questionnaires provided to patients to complete. Hand strength via grip and pinch strength will be assessed using a dynamometer. Hand range of motion will be assessed using a goniometer. Radiographs of the hand (to assess evidence of stability) will be obtained with standard views including anterior-posterior, lateral, and oblique. Each of these assessments will take place in the hand surgery clinic during patients' post-operative follow up visits.

### EXAMPLE 2

### Thumb UCL Ligament Reconstruction

Ten patients with isolated injuries to the thumb UCL, for whom non-operative management has failed, and who have persistent instability, will undergo ligament reconstruction using allograft knee collateral ligaments (i.e., ligament-derived grafts in accordance with the invention described and contemplated herein, “HCL”). Post-operative efficacy will be determined using functional outcome scores, measured strength, range of motion, and radiographs.

The thumb UCL reconstruction technique will be as follows. Dissection is performed over the ulnar aspect of the thumb MCP joint to identify the native UCL. The injured ligament will be debrided, taking care to not disrupt the capsule or volar joint structures. Bicortical drill holes will be made in the metacarpal head and base of the proximal phalanx, at the anatomic attachment sites of the collateral ligament. The prepared HCL is passed through the holes via passing sutures exiting the radial thumb. The graft is secured in the proximal phalanx first using an impedance screw. The graft is tensioned by manually pulling on the passing sutures while maintaining the joint in 30 degrees of flexion. A second impedance screw is used to fix the proximal portion of the graft in the metacarpal head. The reconstructed ligament is sutured to the volar plate for additional stability. Excess graft material and sutures are trimmed.

Efficacy of the foregoing procedure and the HCL (allograft knee collateral ligament) will be assessed via several modalities at the following post-operative time points: 6 weeks, 6 months, and 12 months, and will be compared to pre-operative assessments of the same modalities. DASH (Disabilities of the Arm, Shoulder & Hand) and VAS pain (Visual Analog Scale for pain) scores will be obtained with questionnaires provided to patients to complete. Hand strength via grip and pinch strength will be assessed using a dynamometer. Hand range of motion will be assessed using a goniometer. Radiographs of the hand (to assess evidence of stability) will be obtained with standard views including anterior-posterior, lateral, and oblique. Each of these assessments will take place in the hand surgery clinic during patients' post-operative follow up visits.

### EXAMPLE 3

### SL Ligament Reconstruction

Ten patients with isolated injuries to their wrist SL ligaments, for whom non-operative management has failed, and who have persistent instability will undergo ligament reconstruction using allograft knee collateral ligaments (i.e., ligament-derived grafts in accordance with the invention described and contemplated herein, “HCL”). Post-operative efficacy will be determined using functional outcome scores, measured strength, range of motion, and radiographs.

The SL ligament reconstruction technique will be as follows. The scapholunate ligament is exposed via a dorsal approach to the wrist. The injured SL ligament is debrided. Using the all-dorsal technique for scapholunate reconstruction, drill holes are made in the proximal pole of the scaphoid and centrally at the lunate staying parallel to the SL joint surface. The HCL is secured via interference screw anchors into these holes.

Efficacy of the foregoing procedure and the HCL (allograft knee collateral ligament) will be assessed via several modalities at the following post-operative time points: 6 weeks, 6 months, and 12 months, and will be compared to pre-operative assessments of the same modalities. DASH (Disabilities of the Arm, Shoulder & Hand) and VAS pain (Visual Analog Scale for pain) scores will be obtained with questionnaires provided to patients to complete. Hand strength via grip and pinch strength will be assessed using a dynamometer. Hand range of motion will be assessed using a goniometer. Radiographs of the hand (to assess evidence of stability) will be obtained with standard views including anterior-posterior, lateral, and oblique. Each of these assessments will take place in the hand surgery clinic during patients' post-operative follow up visits

We claim:

1. A ligament-derived graft comprising a portion of a ligament sample recovered from a mammalian donor and having dimensions suitable for use in a surgical procedure to repair a joint of a subject.

2. The ligament-derived graft of claim 1, comprising approximately 45% to 55% of the ligament sample, which was cut lengthwise, approximately in half, to produce two hemi-collateral ligaments, each of which is useful as a ligament-derived graft.

3. The ligament-derived graft of claim 1, wherein the joint is a small to mid-sized joint or a less load bearing joint.

4. The ligament-derived graft of claim 3, wherein the joint is a small joint selected from the group consisting of: an interphalangeal joint, a metacarpophalangeal joint, a radiocarpal joint, an intercarpal joint, a metatarsophalangeal joint, an intermetatarsal joint, a tarsometatarsal joint, a talocrural joint, a subtalar joint, and an inferior tibiofibular joint.

5. The ligament-derived graft of claim 3, wherein the joint is a mid-sized joint or less load bearing joint selected from the group consisting of: a shoulder and an elbow.

6. The ligament-derived graft of claim 1, wherein the ligament-derived graft has a length of from about 50 millimeters (mm) to about 150 mm, and a width of from about 3 mm to about 20 mm.

7. The ligament-derived graft of claim 1, wherein the ligament sample comprised a lateral collateral ligament or a medial collateral ligament.

8. The ligament-derived graft of claim 1, wherein the mammalian donor was a human and the graft is a ligament-derived allograft.

9. A method for repairing a joint of a subject comprising attaching at least one ligament-derived graft according to claim 1, at its ends, to at least two bones of the joint being repaired.

10. The method of claim 9, wherein the step of attaching comprises: attaching a first end of the ligament-derived graft to a first attachment point of a first bone of the joint and attaching a second end of the ligament-derived graft to a second attachment point of a second bone of the joint.

11. The method of claim 10, wherein the first and second attachment points are located on opposite sides of the joint and, after the attaching steps are performed, the ligament-derived graft extends across the joint.

12. The method of claim 9, comprising using at least two ligament-derived grafts and wherein the step of attaching comprises:
attaching a first end of a first ligament-derived graft to a first attachment point of a first bone of the joint;
attaching a second end of the first ligament-derived graft to a second attachment point of a second bone of the joint;
attaching a first end of a second ligament-derived graft to a third attachment point of a third bone of the joint, which may be the same or different from either of the first and second bones of the joint; and
attaching a second end of the second ligament-derived graft to a fourth attachment point of a fourth bone of the joint, which is different from the third bone but may be the same or different from either of the first and second bones of the joint.

13. The method of claim 12, wherein the first and second attachment points are located on opposite sides of the joint and, after the attaching steps are performed, the first ligament-derived graft extends across the joint; and the third and fourth attachment points are located on opposite sides of the joint and, after the attaching steps are performed, the second ligament-derived graft extends across the joint.

14. The method of claim 13, wherein, after the attaching steps are performed, the first ligament-derived graft extends across the joint on a first lateral side of the joint, and the second ligament-derived graft also extends across the joint on the first lateral side of the joint.

15. The method of claim 13, wherein, after the attaching steps are performed, the first ligament-derived graft extends across the joint on a first lateral side of the joint, and the second ligament-derived graft extends across the joint on a second lateral side of the joint, wherein the second lateral side of the joint is opposite the first side thereof.

16. The method of claim 9, further comprising, prior to one or more attaching steps, further resizing of the ligament-derived graft by one or more techniques selected from: stretching the ligament-derived graft, cutting one or both ends of the ligament-derived graft to shorten its length, cutting or shaving one or both edges of the ligament-derived graft to reduce its width, connecting the ligament-derived end-to-end to a second ligament-derived graft or another device or graft to provide additional length.

17. The method of claim 9, further comprising, prior to one or more attaching steps, repositioning or removing at least a portion of a tendon or ligament of the joint.

18. The method of claim 9, wherein the repair of a joint comprises the repair, reinforcement or replacement of one or more ligaments or tendons of the joint.

19. The method of claim 9, wherein the joint is a small to mid-sized joint or a less load bearing joint.

20. The method of claim 19, wherein the joint is a small joint selected from the group consisting of: an interphalangeal joint, a metacarpophalangeal joint, a radiocarpal joint, an intercarpal joint, a metatarsophalangeal joint, an intermetatarsal joint, a tarsometatarsal joint, a talocrural joint, a subtalar joint, and an inferior tibiofibular joint.

21. The method of claim 19, wherein the joint is a mid-sized joint or less load bearing joint selected from the group consisting of: a shoulder and an elbow.

22. A method for producing a ligament-derived graft having dimensions suitable for use in a surgical procedure to repair a joint of a subject, the method comprising the steps of:
obtaining a ligament sample recovered from a mammalian donor; and
cutting the ligament sample lengthwise, through its thickness, to produce at least a first ligament portion and a second ligament portion, at least one of which is useful as a ligament-derived graft in a surgical procedure to repair a joint.

23. The method of claim 22, wherein the first ligament portion comprises approximately 40% to 70% of the ligament sample, and the second ligament portion comprises approximately 30% to 60% of the ligament sample.

24. The method of claim 23, wherein the step of cutting comprises cutting the ligament sample approximately in half, lengthwise and through its thickness, wherein the first ligament portion is a first hemi-ligament comprising approximately 45% to 55% of the ligament sample, and the second ligament portion is a second hemi-ligament comprising approximately 45% to 55% of the ligament sample.

25. The method of claim 22, wherein at least one of the first ligament portion and the second ligament portion has dimensions suitable for use in a surgical procedure to repair a joint of a subject.

26. The method of claim 25, wherein the dimensions comprise a length of from about 50 mm to about 150 mm, and a width of from about 3 mm to about 20 mm.

27. The method of claim 22, wherein the ligament sample comprises a lateral collateral ligament or a medial collateral ligament.

28. The method of claim 22, further comprising, before or after the step of cutting, subjecting the ligament segment, or a portion thereof, to one or more processing steps selected from: cleaning; disinfecting; sterilizing; further resizing; contacting with one or more components selected from preservation media, culture media, another biocompatible fluid, a therapeutic material, cells or cell components; and packaging for storage, shipping or both.

29. The method of claim 22, wherein the joint is a small to mid-sized joint or a less load bearing joint.

30. The method of claim 29, wherein the joint is a small joint selected from the group consisting of: an interphalangeal joint, a metacarpophalangeal joint, a radiocarpal joint, an intercarpal joint, a metatarsophalangeal joint, an intermetatarsal joint, a tarsometatarsal joint, a talocrural joint, a subtalar joint, and an inferior tibiofibular joint.

31. The method of claim 29, wherein the joint is a mid-sized joint or less load bearing joint selected from the group consisting of: a shoulder and an elbow.
